(19) United States
(12) Patent Application Publication
(10) Pub. No.: US 2023/0016754 A1
(43) Pub. Date: Jan. 19, 2023

(54) SYSTEM AND APPARATUS FOR ANATOMY STATE CONFIRMATION IN SURGICAL ROBOTIC ARM

(71) Applicant: Covidien LP, Mansfield, MA (US)

(72) Inventor: Dwight Meglan, Westwood, MA (US)

(21) Appl. No.: 17/783,434

(22) PCT Filed: Nov. 12, 2020
(86) PCT No.: PCT/US2020/060166

§ 371 Date: Jun. 8, 2022

Related U.S. Application Data
(60) Provisional application No. 62/945,370, filed on Dec. 9, 2019.

(51) Int. Cl. A61B 34/35; A61B 34/37; A61B 34/00; A61B 34/20; B25J 9/16

(52) U.S. Cl. CPC A61B 34/35; A61B 34/37; A61B 34/25; A61B 34/20; B25J 9/161; B25J 9/163; A61B 2034/2057; A61B 2034/2065

(57) ABSTRACT

A surgical robotic system includes a surgical console having a display and a user input device configured to generate a user input and a surgical robotic arm, which includes a surgical instrument configured to treat tissue and being actuatable in response to the user input and a video camera configured to capture video data that is displayed on the display. The system also includes a control tower coupled to the surgical console and the surgical robotic arm. The control tower is configured to process the user input to control the surgical instrument and to record the user input as input data; communicate the input data and the video data to at least one machine learning system configured to generate a surgical process evaluator; and execute the surgical process evaluator to determine whether the surgical instrument is properly positioned relative to the tissue.

### BACKGROUND

Surgical robotic systems are currently being used in minimally invasive medical procedures. Some surgical robotic systems may include a surgical console controlling a surgical robotic arm and a surgical instrument having an end effector (e.g., forceps or grasping tool) coupled to and actuated by the robotic arm.

During laparoscopic robotic surgical procedures tissue may be manipulated in a variety of ways, such as vessel sealing through energy application, tissue re-approximation, and/or sealing and separation using a stapler. These actions are difficult to undo should the action to be performed incorrectly or at the wrong location. Accordingly, there is a need for a system that is configured to check positioning of the surgical instrument and confirm that the placement is correct prior to the actuation of the surgical instrument, including application of energy, deployment of staples, and separation of tissue.

### SUMMARY

The present disclosure provides a surgical robotic system including a first machine learning algorithm that allows the system to determine a point in the procedure that has been reached during which a surgical instrument can be activated, namely, whether energy may be applied through an electrosurgical forces and/or staples may be deployed through a stapler. The system also includes a second machine learning algorithm that allows the system to determine tissue geometry relative to the vessel sealer or stapler. The system is further configured to automatically, e.g., using a processor, determine whether the surgical instrument can be actuated and/or fired. If the system determines that the surgical instrument cannot be actuated, the system is further configured to prevent actuation of the surgical instrument and to provide feedback as to why the system has made this judgement including indicating on a display, e.g., an endoscope video image, location of the problem and the reason for the problem. When the surgical instrument is actuated, the system also records information about the surgical instrument deployment including the state of the tissue. The system according to the present disclosure utilizes discernment automation to confirm correct positioning of the surgical instrument, proper sequence operation of the surgical instrument during the procedure, before actuating the surgical instrument and confirm that the surgical instrument has been properly actuated.

According to one embodiment of the present disclosure, a surgical robotic system is disclosed. The system includes a surgical console having a display and a user input device configured to generate a user input and a surgical robotic arm, which includes a surgical instrument configured to treat tissue and being actuatable in response to the user input and a video camera configured to capture video data that is displayed on the display. The system also includes a control tower coupled to the surgical console and the surgical robotic arm. The control tower is configured to process the user input to control the surgical instrument and to record the user input as input data; communicate the input data and the video data to at least one machine learning system configured to generate a surgical process evaluator; and execute the surgical process evaluator to determine whether the surgical instrument is properly positioned relative to the tissue.

According to one aspect of the above embodiment, the at least one machine learning system is a neural network. The neural network is trained using at least one of supervised training, unsupervised training, or reinforcement learning.

According to another aspect of the above embodiment, the surgical process evaluator is configured to determine whether the tissue is properly disposed within the surgical instrument. The surgical process evaluator is further configured to prevent actuation of the surgical instrument in response to the surgical process evaluator determining that the tissue is not properly disposed within the surgical instrument. The surgical process evaluator is also configured to output at least one of an audio or video indication in response to the surgical process evaluator determining that the tissue is not properly disposed within the surgical instrument.

According to another embodiment of the present disclosure, a surgical robotic system is disclosed. The system includes: a surgical console having a display and a user input device configured to generate a user input and a surgical robotic arm having a surgical instrument configured to treat tissue and being actuatable in response to the user input and a video camera configured to capture video data that is displayed on the display. The system also includes a control tower coupled to the surgical console and the surgical robotic arm, the control tower configured to: execute a procedure progress evaluator configured to determine progress of a surgical procedure and execute a surgical site tool use evaluator configured to determine whether the surgical instrument is properly positioned relative to the tissue.

According to one aspect of the above embodiment, the control tower is configured to process the user input to control the surgical instrument and to record the user input as input data. The control tower is also configured to communicate the input data and the video data to a first machine learning system and a second machine learning system. The first machine learning system and the second machine learning system may be neural networks. The neural networks are trained using at least one of supervised training, unsupervised training, or reinforcement learning.

According to another aspect of the above embodiment, the surgical site tool use evaluator is configured to determine whether the tissue is properly disposed within the surgical instrument. The surgical site tool use evaluator is further configured to prevent actuation of the surgical instrument in response to the surgical site tool use evaluator determining that the tissue is not properly disposed within the surgical instrument. The surgical site tool use evaluator is also configured to output at least one of an audio or video indication in response to the surgical site tool use evaluator determining that the tissue is not properly disposed within the surgical instrument.

According to a further embodiment of the present disclosure, a method for controlling a surgical robotic system is disclosed. The method includes: generating a user input through a user input device of a surgical console; and processing the user input to generate a movement command at a control tower coupled to the surgical console. The method also includes transmitting the movement command to a surgical robotic arm, the surgical robotic arm including a surgical instrument configured to treat tissue and being actuatable in response to the user input. The method further includes capturing video data through a video camera disposed on the surgical robotic arm; and communicating the user input and the video data to at least one machine learning system. The method includes generating, using the at least one machine learning system, a surgical process evaluator; and executing the surgical process evaluator to determine whether the surgical instrument is properly positioned relative to the tissue.

According to one aspect of the above embodiment, the at least one machine learning system is a neural network. The method further includes training the neural network using at least one of supervised training, unsupervised training, or reinforcement learning.

According to another aspect of the above embodiment, the method further includes determining whether the tissue is properly disposed within the surgical instrument using the surgical process evaluator. The method also includes preventing actuation of the surgical instrument in response to the surgical process evaluator determining that the tissue is not properly disposed within the surgical instrument. The method further includes outputting at least one of an audio or video indication in response to the surgical process evaluator determining that the tissue is not properly disposed within the surgical instrument.

### BRIEF DESCRIPTION OF THE DRAWINGS

Embodiments of the present disclosure are described herein with reference to the accompanying drawings, wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a schematic diagram of first and second machine learning systems implemented in the surgical robotic system of FIG. 1 to an embodiment of the present disclosure; and

FIG. 6 is a flow chart of a method according to the present disclosure utilizing algorithms based on the first and second machine learning systems of FIG. 5 to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

As will be described in detail below, the present disclosure is directed to a surgical robotic system, which includes a surgical console, a control tower, and one or more mobile carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

The terms “artificial intelligence,” “data models,” or “machine learning” may include, but are not limited to, neural networks, convolutional neural networks (CNN), recurrent neural networks (RNN), generative adversarial networks (GAN), Bayesian Regression, Naive Bayes, nearest neighbors, least squares, means, and support vector regression, among other data science and artificial science techniques.

The term “application” may include a computer program designed to perform functions, tasks, or activities for the benefit of a clinician. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

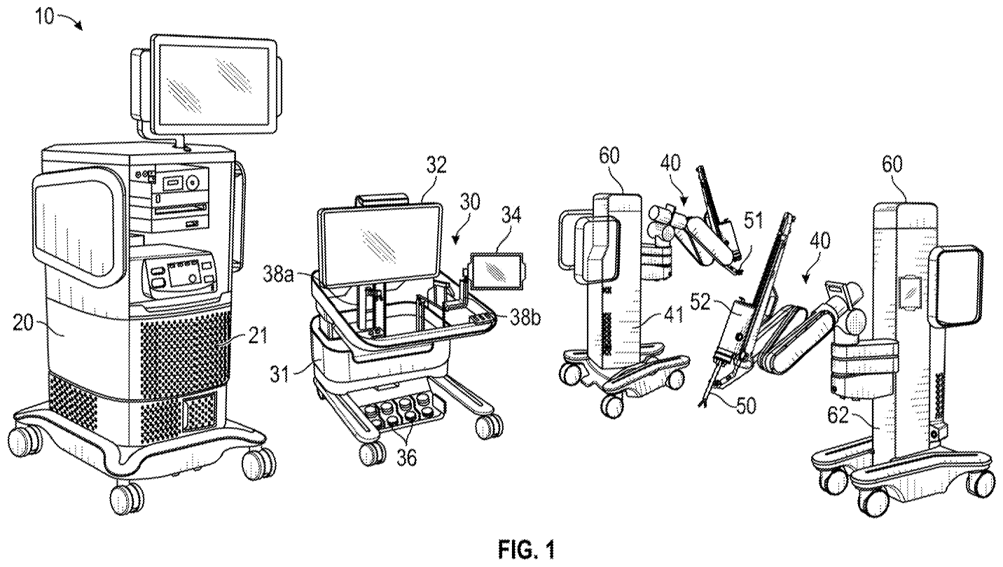

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope configured to provide a video feed for the clinician. In further embodiments, the surgical instrument 50 may be an electrosurgical forceps configured to seal tissue by compression tissue between jaw members and applying electrosurgical current thereto. In yet further embodiments, the surgical instrument 50 may be a surgical stapler including a pair of jaws configured to grasp and clamp tissue whilst deploying a plurality of tissue fasteners, e.g., staples, and cutting stapled tissue.

Each of the robotic arms 40 may include a camera 51 configured to capture video of the surgical site. The camera 51 may be disposed along with the surgical instrument 50 on the robotic arm 40. The surgical console 30 includes a first display 32, which displays a video feed of the surgical site provided by camera 51 of the surgical instrument 50 disposed on the robotic arms 40, and a second display device 34, which displays a user interface for controlling the surgical robotic system 10. The surgical console 30 also includes a plurality of user interface devices, such as foot pedals 36 and a pair of handle controllers 38a and 38b which are used by a clinician to remotely control robotic arms 40.

The control tower 20 acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

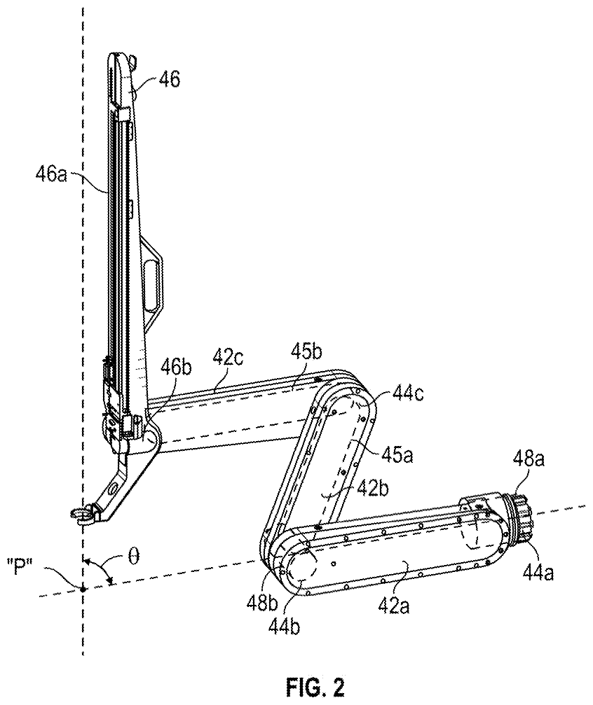

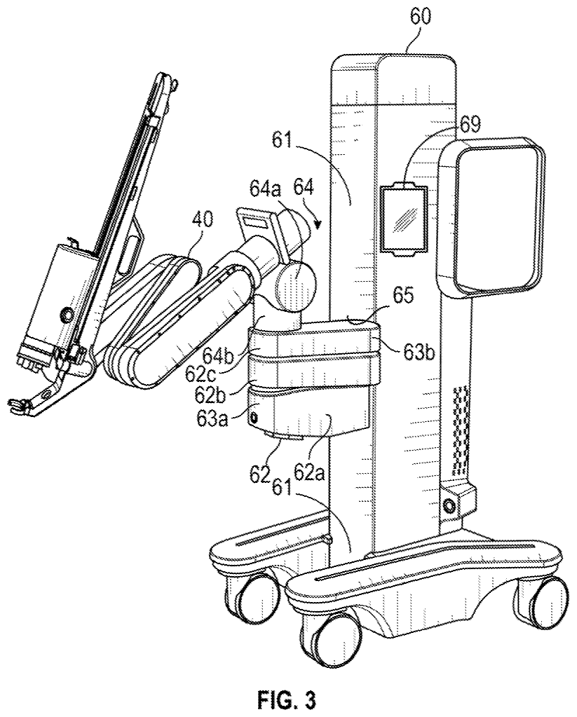

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference to FIG. 2, the robotic arm 40 also includes a holder 46 defining a second longitudinal axis and configured to receive an instrument drive unit 52 (FIG. 1) of the surgical instrument 50, which is configured to couple to an actuation mechanism of the surgical instrument 50. Instrument drive unit 52 transfers actuation forces from its actuators to the surgical instrument 50 to actuate components (e.g., end effectors) of the surgical instrument 50. The holder 46 includes a sliding mechanism 46a, which is configured to move the instrument drive unit 52 along the second longitudinal axis defined by the holder 46. The holder 46 also includes a joint 46b, which rotates the holder 46 relative to the link 42c.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

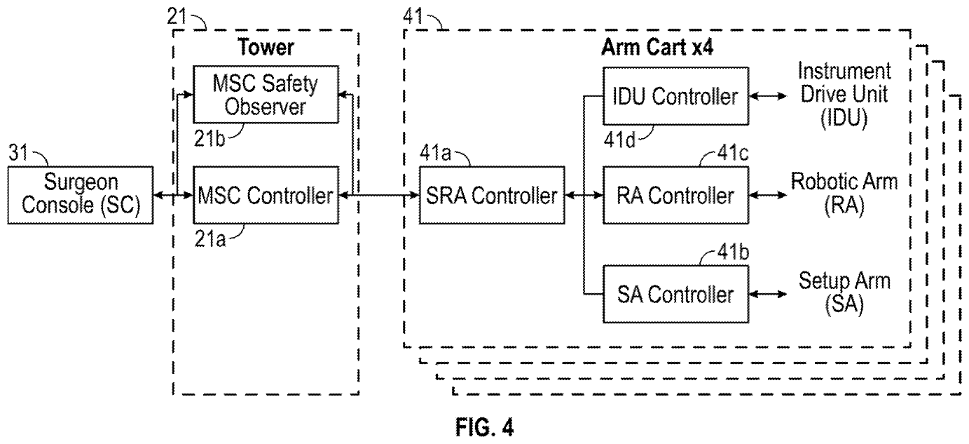

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

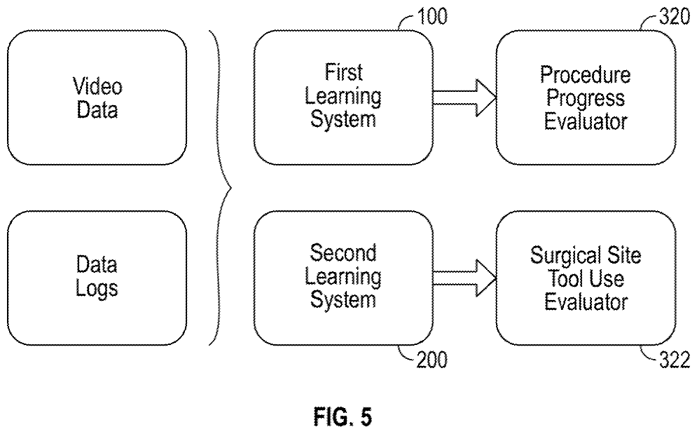

With reference to FIG. 5, the surgical robotic system 10 includes a first learning system 100 and a second learning system 200. The first learning system 100 and the second learning system 200 may be neural networks. In various embodiments, the neural networks may include a temporal convolutional network, with one or more fully connected layers, or a feed forward network. In various embodiments, training of the neural networks may happen on a separate system, e.g., graphic processor unit (“GPU”) workstations, high performing computer clusters, etc., and the trained networks would then be deployed in the surgical robotic system 10. In further embodiments, training of the neural networks may happen locally, e.g., on the computer 21.

The first learning system 100 and the second learning system 200 receive as input video data from the camera(s) 51 of the surgical instrument 50. The video and data logs from prior surgical procedures may be used to train the first learning system 100 and the second learning system 200.

The first learning system 100 generates a procedure progress evaluator 320 based on the input data and training. The procedure progress evaluator 320 is configured to discern progress in the specific procedure being performed using the surgical instrument 50. The second learning system 200 is configured to generate a surgical site tool use evaluator 322 based on the input data and training. The surgical site tool use evaluator 322 is configured discern the specific details of how the surgical instrument 50 is about to be used. The procedure progress evaluator 320 and the surgical site tool use evaluator 322 may be embodied as an application or software executable by the computer 21 of the control tower 20.

The procedure progress evaluator 320 initially receives input regarding the type of the surgical instrument 50 (e.g., electrosurgical forceps, tissue stapler, etc.), the type and location of a tissue site on which procedure is being performed, as well as the specific type of the procedure. The procedure progress evaluator 320 may also include a timer to keep track of time since commencement of the procedure. In addition, the procedure progress evaluator 320 includes an event tracker for each command issued from the surgical console 30. The procedure progress evaluator 320 incorporates the video input, time, and event tracker to determine at what specific stage of the procedure the surgical instrument 50 is being used. In embodiments, opening of jaw members (e.g., vessel sealer or a stapler) of the surgical instrument 50 are interpreted by the first learning system 100 as being indicative of tissue about to be grasped. The first learning system 100 may also include other conditions, such as location of the surgical instrument 50 (e.g., within or outside the patient, relative to a specific organ or anatomical structure, etc.), for determining when grasping of tissue or other actuations of the surgical instrument 50 are about to occur.

The procedure progress evaluator 320 and the surgical site tool use evaluator 322 may be used in concert with one another. The procedure progress evaluator 320 provides the overall context for the specific action in relation to the entire procedure. The surgical site tool use evaluator 322 evaluates the specific action, e.g., use of the surgical instrument 50. Following this approach, the procedure progress evaluator 320 accesses the data, namely, video input, user input, movement and position of the surgical instrument 50, to appropriately analyze the placement of the surgical instrument 50 relative to the tissue that is being operated upon, which may include determining whether the tissue is disposed within the surgical instrument 50, e.g., grasped by the jaws, whether certain anatomical regions are outside the surgical instrument 50.

The second learning system 200 is trained with the same data as the first learning system 100 but could also be trained with additional video and user inputs focused solely on the time prior to the use of the surgical instrument 50. The second learning system 200 is configured to generate a surgical site tool use evaluator 322 based on the input data and training. The surgical site tool use evaluator 322 is configured discern the specific details of how the surgical instrument 50 is about to be used. In particular, the surgical site tool use evaluator 322 is focused on specific details of the anatomy surrounding the surgical instrument 50 and may track and prevent poor usage of the surgical instrument 50 including prevention of mistakes.

The procedure progress evaluator 320 and the surgical site tool use evaluator 322 may also provide detailed annotations during procedures on the first display 32 to enable the first learning system 100 and the second learning system 200 to learn to a sufficient level of detail to provide clinically useful guidance during subsequent procedures.

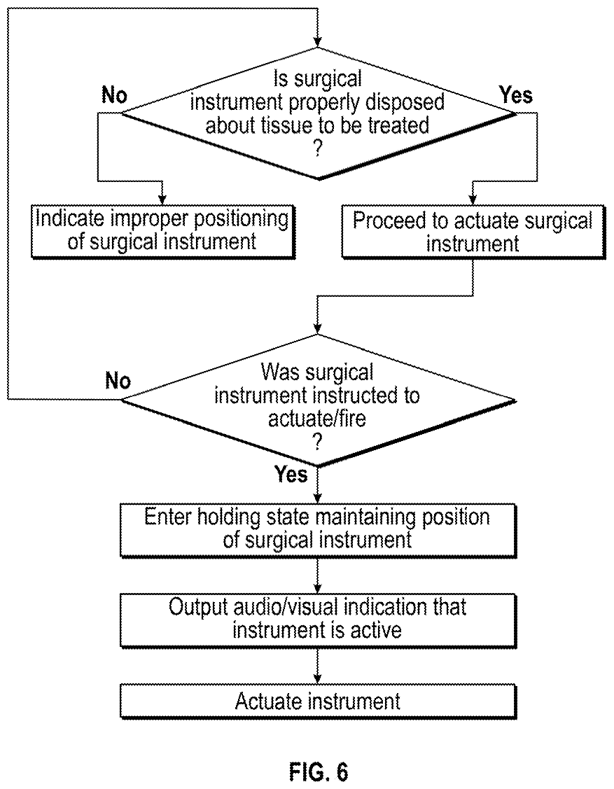

With reference to FIG. 6, a method implementing procedure progress evaluator 320 and the surgical site tool use evaluator 322 is disclosed. The system 10 utilizes procedure progress evaluator 320 and the surgical site tool use evaluator 322 to determine whether the tissue is properly disposed within and/or about the surgical instrument 50 after the placing the surgical instrument 50 into the steady state. If the system 10 determines that the tissue is properly disposed within the surgical instrument 50 to effect appropriate vessel sealing or staple line creation, then the system 10 actuates to the surgical instrument 50. If the system 10 does not detect a proper placement, the system 10 visually and/or audibly indicates to the clinician in the endoscopic view of the first display 32 that use of the surgical instrument 50 is not proper along with reasons why the surgical instrument 50 is prevented from actuation as well as provide guidance as to how to readjust the surgical instrument 50 relative to the tissue to allow proper use.

The computer 21 detects that the surgical instrument 50 has been instructed by the clinician to actuate/fire via the surgical console 30 using the procedure progress evaluator 320 and the surgical site tool use evaluator 322. If the computer 21 determines that the surgical instrument 50 is about to be actuated, the computer 21 enters a holding state during which the robotic arm 40 is held steady to ensure that the surgical instrument 50 is stable during use. The system 10 may also output a visual and/or audio indication to alert the clinician that the surgical instrument 50 is active so that the clinician can be mindful of holding steady the surgical instrument 50.

Once the surgical instrument 50 has been actuated, the system 10 releases the robotic arm 40. The actuation process as well as the state of the surgical site post actuation is recorded by the system 10, including video and logging data, and combined with available surgical instrument 50 state data to provide a permanent record that can be used for documentation and/or for future refinement of use of the surgical instrument 50 as well as for additional training of the first learning system 100 and the second learning system 200.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a surgical console including a display and a user input device configured to generate a user input;
a surgical robotic arm including:
a surgical instrument configured to treat tissue and being actuatable in response to the user input; and
a video camera configured to capture video data that is displayed on the display; and
a control tower coupled to the surgical console and the surgical robotic arm, the control tower configured to:
process the user input to control the surgical instrument and to record the user input as input data;
communicate the input data and the video data to at least one machine learning system configured to generate a surgical process evaluator; and
execute the surgical process evaluator to determine whether the surgical instrument is properly positioned relative to the tissue.

2. The surgical robotic system according to claim 1, wherein the at least one machine learning system is a neural network.

3. The surgical robotic system according to claim 2, wherein the neural network is trained using at least one of supervised training, unsupervised training, or reinforcement learning.

4. The surgical robotic system according to claim 1, wherein the surgical process evaluator is configured to determine whether the tissue is properly disposed within the surgical instrument.

5. The surgical robotic system according to claim 4, wherein the surgical process evaluator is configured to prevent actuation of the surgical instrument in response to the surgical process evaluator determining that the tissue is not properly disposed within the surgical instrument.

6. The surgical robotic system according to claim 4, wherein the surgical process evaluator is configured to output at least one of an audio or video indication in response to the surgical process evaluator determining that the tissue is not properly disposed within the surgical instrument.

7. A surgical robotic system comprising:
a surgical console including a display and a user input device configured to generate a user input;
a surgical robotic arm including:
a surgical instrument configured to treat tissue and being actuatable in response to the user input; and
a video camera configured to capture video data that is displayed on the display; and
a control tower coupled to the surgical console and the surgical robotic arm, the control tower configured to:
execute a procedure progress evaluator configured to determine progress of a surgical procedure; and
execute a surgical site tool use evaluator configured to determine whether the surgical instrument is properly positioned relative to the tissue.

8. The surgical robotic system according to claim 7, wherein the control tower is configured to process the user input to control the surgical instrument and to record the user input as input data.

9. The surgical robotic system according to claim 8, wherein the control tower is configured to communicate the input data and the video data to a first machine learning system and a second machine learning system.

10. The surgical robotic system according to claim 9, wherein the first machine learning system and the second machine learning system are neural networks.

11. The surgical robotic system according to claim 10, wherein the neural networks are trained using at least one of supervised training, unsupervised training, or reinforcement learning.

12. The surgical robotic system according to claim 7, wherein the surgical site tool use evaluator is configured to determine whether the tissue is properly disposed within the surgical instrument.

13. The surgical robotic system according to claim 12, wherein the surgical site tool use evaluator is configured to prevent actuation of the surgical instrument in response to the surgical site tool use evaluator determining that the tissue is not properly disposed within the surgical instrument.

14. The surgical robotic system according to claim 13, wherein the surgical site tool use evaluator is configured to output at least one of an audio or video indication in response to the surgical site tool use evaluator determining that the tissue is not properly disposed within the surgical instrument.

15. A method for controlling a surgical robotic system, the method comprising:
generating a user input through a user input device of a surgical console;
processing the user input to generate a movement command at a control tower coupled to the surgical console;
transmitting the movement command to a surgical robotic arm, the surgical robotic arm including a surgical instrument configured to treat tissue and being actuatable in response to the user input;
capturing video data through a video camera disposed on the surgical robotic arm;
communicating the user input and the video data to at least one machine learning system;
generating, using the at least one machine learning system, a surgical process evaluator; and
executing the surgical process evaluator to determine whether the surgical instrument is properly positioned relative to the tissue.

16. The method according to claim 15, wherein the at least one machine learning system is a neural network.

17. The method according to claim 16, further comprising: training the neural network using at least one of supervised training, unsupervised training, or reinforcement learning.

18. The method according to claim 15, further comprising: determining whether the tissue is properly disposed within the surgical instrument using the surgical process evaluator.

19. The method according to claim 18, further comprising: preventing actuation of the surgical instrument in response to the surgical process evaluator determining that the tissue is not properly disposed within the surgical instrument.

20. The method according to claim 19, further comprising: outputting at least one of an audio or video indication in response to the surgical process evaluator determining that the tissue is not properly disposed within the surgical instrument.
